SEQUENCE LISTING (12 sequences)

SEQ ID NO: 1 - protein, 255 aa, synthetic construct
  REGION 1..255: note = Synthetic Construct
ASQVQLVQSGAEVKNPGASVKVSCKASGYAFTSYGISWVRQAPGQGLEWMGWISAYTRNTNYAQKFQGRVTLTTDTSTSTAYMELRSLRSDDTAIYYCARDARYSLGVGAFDVWGQGTMVTVSSGILGSGGGGSGGGGSGGGGSETTLTQSPAFMSATPGDKVSISCKASRDIDDDLNWYQQKPGEAAISIIQEATTLVPGIPPRFSGSGYGTDFTLTINNIESEDAAYYFCLQHDDVPYTFGQGTKLEIKSGIL

SEQ ID NO: 2 - protein, 257 aa, synthetic construct
  REGION 1..257: note = Synthetic Construct
ASQITLKESGPTLVKPTQTLTLTCSFSGFSLSFSGVGVAWIRQPPGKALEWLALIYWDDDKRYSPSLKSRLTITKDTSKNQVVLTMTNMDPLDTATYYCAHREWKAFGAFDIWGQGTMVTVSSGILGSGGGGSGGGGSGGGGSQPVLTQSPSASGTPGQRVTISCSGSSSNIGSNTVNWYQQLPGTAPKLLIYSNNQRPSGVPDRFSGSKSVTSASLAISGLQSEDEAEYYCATWDDSLNGAVFGGGTQLTVLSGIL

SEQ ID NO: 3 - protein, 127 aa, synthetic construct
  REGION 1..127: note = Synthetic Construct
ASQVQLVQSGAEVKNPGASVKVSCKASGYAFTSYGISWVRQAPGQGLEWMGWISAYTRNTNYAQKFQGRVTLTTDTSTSTAYMELRSLRSDDTAIYYCARDARYSLGVGAFDVWGQGTMVTVSSGIL

SEQ ID NO: 4 - protein, 114 aa, synthetic construct
  REGION 1..114: note = Synthetic Construct
ETTLTQSPAFMSATPGDKVSISCKASRDIDDDLNWYQQKPGEAAISIIQEATTLVPGIPPRFSGSGYGTDFTLTINNIESEDAAYYFCLQHDDVPYTFGQGTKLEIKSGILSEQ

SEQ ID NO: 5 - protein, 129 aa, synthetic construct
  REGION 1..129: note = Synthetic Construct
ASQITLKESGPTLVKPTQTLTLTCSFSGFSLSFSGVGVAWIRQPPGKALEWLALIYWDDDKRYSPSLKSRLTITKDTSKNQVVLTMTNMDPLDTATYYCAHREWKAFGAFDIWGQGTMVTVSSGILSEQ

SEQ ID NO: 6 - protein, 114 aa, synthetic construct
  REGION 1..114: note = Synthetic Construct
QPVLTQSPSASGTPGQRVTISCSGSSSNIGSNTVNWYQQLPGTAPKLLIYSNNQRPSGVPDRFSGSKSVTSASLAISGLQSEDEAEYYCATWDDSLNGAVFGGGTQLTVLSGIL

SEQ ID NO: 7 - protein, 126 aa, synthetic construct
  REGION 1..126: note = Synthetic Construct
QVQLQESGGGLVQAGGSLRLSCAASGNISYFLIMGWYRQAPGKEREFVAAITRGSNTYYADSVKGRFTISRDNAKNTVYLQMNSLKPEDTAVYYCAVFSTLQYHYDTGYTAYLTYWGQGTQVTVSS

SEQ ID NO: 8 - protein, 7 aa, synthetic construct
  REGION 1..7: note = Synthetic Construct
NISYFLI

SEQ ID NO: 9 - protein, 14 aa, synthetic construct
  REGION 1..14: note = Synthetic Construct
EFVAAITRGSNTYY

SEQ ID NO: 10 - protein, 20 aa, synthetic construct
  REGION 1..20: note = Synthetic Construct
AVFSTLQYHYDTGYTAYLTY

SEQ ID NO: 11 - protein, 5 aa, synthetic construct
  REGION 1..5: note = Synthetic Construct
GGGGS

SEQ ID NO: 12 - protein, 5 aa, synthetic construct
  REGION 1..5: note = Synthetic Construct
EAAAK

(19) United States
(12) Patent Application Publication
(10) Pub. No.: US 2025/0297033 A1
(43) Pub. Date: Sep. 25, 2025

(54) BISPECIFIC ANTIBODY FOR MEMBRANE CLEARANCE OF TARGET RECEPTORS

(71) Applicant: H. Lee Moffitt Cancer Center and Research Institute, Inc., Tampa, FL (US)

(72) Inventor: Vincent C. Luca, Tampa, FL (US)

(21) Appl. No.: 19/229,563

(22) Filed: Jun. 5, 2025

Related U.S. Application Data
(63) Continuation of application No. 17/311,080, filed on Jun. 4, 2021.
(60) Provisional application No. 62/785,451, filed on Dec. 27, 2018.

(51) Int. Cl. C07K 16/40; C07K 16/28

(52) U.S. Cl. CPC C07K 16/40; C07K 16/28; C07K 2317/20; C07K 2317/31; C07K 2317/34; C07K 2317/569; C07K 2317/622; C07K 2317/92

(57) ABSTRACT

Disclosed are bispecific molecules, referred to herein as ubiquibodies, that are able to ubiquitinate target cell surface receptors on a target cell. The ubiquibodies can be engineered from fusion polypeptides comprising 1) variable domains of antibodies that specifically bind a target cell surface receptor and 2) variable domains of antibodies that specifically bind a transmembrane E3 ubiquitin ligase (TMUL). Either or both components of the ubiquibodies can also be engineered from non-antibody scaffolds including but not limited to nanobodies, monobodies, cyclic peptides, small molecules, and designed ankyrin repeat proteins (Darpins).

### CROSS-REFERENCE TO RELATED APPLICATIONS

This application is a continuation of copending application Ser. No. 17/311,080, filed Jun. 4, 2021, which is a National Stage of International Application No. PCT/US2019/068585, filed Dec. 26, 2019, which claims benefit of U.S. Provisional Application No. 62/785,451, filed Dec. 27, 2018, which is hereby incorporated herein by reference in its entirety.

### SEQUENCE LISTING

This application contains a sequence listing filed in ST.26 format entitled “320803-1871 Sequence Listing” created on May 14, 2025, and having 16,888 bytes. The content of the sequence listing is incorporated herein in its entirety.

### BACKGROUND

The behavior and identity of a given cell is largely dictated by the specific landscape of receptors presented on its surface. Receptor homeostasis is critical for normal cellular function, and aberrant receptor expression contributes to the pathogenesis of cancer, viral infection, autoimmunity and a myriad of other devastating diseases. Although several drugs antagonize receptor function through steric inhibition, the development of agents that modulate the receptor landscape remains a major challenge in molecular pharmacology. Soluble ligands capable of “dialing down” receptor levels would have transformative therapeutic potential for a broad spectrum of human diseases and would undoubtedly serve as powerful tools for basic research.

### SUMMARY

Disclosed are bispecific molecules, referred to herein as ubiquibodies, that are able to ubiquitinate target cell surface receptors on a target cell. The ubiquibodies can be engineered from fusion polypeptides comprising 1) variable domains of antibodies that specifically bind a target cell surface receptor and 2) variable domains of antibodies that specifically bind a transmembrane E3 ubiquitin ligase (TMUL). Either or both components of the ubiquibodies can also be engineered from non-antibody scaffolds including but not limited to nanobodies, monobodies, cyclic peptides, small molecules, and designed ankyrin repeat proteins (Darpins).

The TMUL can in some embodiments be any protein of a target cell that possess an extracellular domain (ECD), a transmembrane domain (TMD), and an intracellular domain (ICD), wherein the ICD contains a RING E3 domain. When the bispecific antibody simultaneously binds the ECD of the TMUL and the target receptor, it catalyzes ubiquitination of the target receptor. Examples of known TMULs that can be used to ubiquitinate target receptors include ZNRF3, RNF43, GRAIL (RNF128), RNF13, RNF148, RNF149, RNF150, RNF167, RNF133, Goliath, RNF150, RNF122, ZNRF4, Gp78, HRD1, RNF170, RNF121, RNF175, TRC8, RNF145, MARCH5, ZFPL1, RNFT1, RINES, Kf-1, RNF182, RMA1, RNF185, RNF19, RNF144, RNF217, MARCH1, MARCH8, MARCH2, MARCH3, MARCH11, MARCH4, MARCH9, MARCH6, BAR, RNF26, DCST1, RNF152, RNF183, RNF186, RNF197, MAPL, TRIM13, TRIM59, and ZNF179.

In some embodiments, the antibody is a diabody (fusion polypeptide) having, for example, the following formula:

V_{L}R-V_{H}T & V_{L}T-V_{H}R, or

V_{H}R-V_{L}T & V_{H}T-V_{L}R,
wherein “V_{L}R” is a light chain variable domain specific for an target cell surface receptor;
wherein “V_{H}T” is a heavy chain variable domain specific for a TMUL;
wherein “V_{L}T” is a light chain variable domain specific for the TMUL;
wherein “VHR” is a heavy chain variable domain specific for the target cell surface receptor; and
wherein “-” consists of a peptide linker or a peptide bond.

In some embodiments, the antibody is a Bispecific T-Cell Engaging (BiTE) antibody (fusion polypeptide) having, for example, the following formula:

V_{L}R-V_{H}R-V_{L}T-V_{H}T,

V_{H}R-V_{L}R-V_{H}T-V_{L}T,

V_{L}R-V_{H}R-V_{H}T-V_{L}T, or

V_{H}R-V_{L}R-V_{L}T-V_{H}T,
wherein “V_{L}R” is a light chain variable domain specific for an target cell surface receptor;
wherein “V_{H}T” is a heavy chain variable domain specific for a TMUL;
wherein “V_{L}T” is a light chain variable domain specific for the TMUL;
wherein “V_{H}R” is a heavy chain variable domain specific for the target cell surface receptor; and
wherein “-” consists of a peptide linker or a peptide bond.

In some embodiments, the antibody is a Bispecific having, for example, the following formula:

V_{H}R-V_{H}T,

V_{H}T-V_{H}R,

V_{H}R-V_{H}T-V_{L}T,

V_{H}R-V_{L}T-V_{H}T,

V_{H}R-V_{L}R-V_{H}T, or

V_{L}R-V_{H}R-V_{H}T,
wherein “V_{L}R” is a light chain variable domain specific for an target cell surface receptor;
wherein “V_{H}T” is a heavy chain variable domain specific for a TMUL;
wherein “V_{L}T” is a light chain variable domain specific for the TMUL;
wherein “V_{H}R” is a heavy chain variable domain specific for the target cell surface receptor; and
wherein “-” consists of a peptide linker or a peptide bond.

In some embodiments, the antibody is a bispecific antibody containing the full heavy and light chain regions. In this embodiment, the antibody may be generated by described methods such as the “knobs and holes” format (published in Ridgway J B, et al, Protein Eng. 1996 9 (7): 617-21).

The target cell surface receptor of the disclosed compositions and methods is not a receptor that binds an R-spondin protein and is therefore naturally ubiquitinated by a TMUL, such as a leucine-rich repeat-containing G-protein coupled receptor (LGR). The target cell surface receptor can in some cases be any other cell surface receptor, channel, or transporter that contains lysine residues in its intracellular domain and is expressed on a target cell that also expresses a TMUL. The receptor is preferably a receptor associated with a disease or disorder. In some embodiments, the receptor is an immune checkpoint, such as PD-L1 or CD86. In some embodiments, the receptor is an innate/adaptive immune receptor such as IFNAR, IL-2RG, or MHC class I. In some embodiments, the receptor is an HIV receptor such as CD4 or CXCR4. In some embodiments, the receptor is an oncogenic receptor such as Smo, EGFR, or HER2. In some embodiments, the receptor is an inflammatory/autoimmune receptor such as TNFR1 or NDMA-R. Other disease associated membrane proteins that may be targeted include GPCRs, cytokine receptors, Notch receptors, receptor tyrosine kinases, MHC class II, calcium channels, TGF-beta family receptors, NF-KappaB receptors, cadherins, integrins or any other transmembrane protein that contains lysines in the intracellular region. In some embodiments, the receptor is any cell surface receptor that has lysine residues in its intracellular domain.

In some embodiments, the receptor is a tumor associated antigen (TAA). Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gpIOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvlll, IL-13Ra, CD123, CD19, CD33, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

Also disclosed is an isolated nucleic acid encoding the disclosed fusion polypeptide, as well as nucleic acid vectors containing this isolated nucleic acid operably linked to an expression control sequence. Also disclosed are cells transfected with these vectors and the use of these cells to produce the disclosed fusion polypeptides.

A bi-specific antigen binding molecule can be formed from dimerization of heavy and light chains. In these embodiments, the V_{L}R dimerizes with VAR to form an antigen binding site for a target cell surface receptor and the V_{H}T dimerizes with V_{L}T to form an antigen binding site for a TMUL.

Also disclosed is a bispecific antibody that is a single polypeptide chain comprising a bispecific antibody having a first antigen-binding region and a second antigen-binding region. In some cases, the first antigen-binding region is capable of specifically binding to the target receptor on the cell; and the second antigen-binding region is capable of specifically binding to a TMUL on the cell.

Each of the first and second portions can comprise 1, 2, 3, or more antibody variable domains. In particular embodiments, each of the first and second portions contains two variable domains, a variable heavy (V_{H}) domain and a variable light (V_{L}) domain.

In some cases, the bispecific antibody has an affinity for the target receptor and the TMUL corresponding to a K_{D} of about 10⁻⁷ M, 10⁻⁸ M, 10⁻⁹ M, or less.

Each of the first and second portions can be derived from natural antibodies, such as monoclonal antibodies. In some cases, the antibody is human. In some cases, the bispecific antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Currently, the most widely used technique for antibody human adaptation is known as “CDR grafting.” The scientific basis of this technology is that the binding specificity of an antibody resides primarily within the three hypervariable loops known as the complementarity determining regions (CDRs) of its light and heavy chain variable regions (V-regions), whereas the more conserved framework regions (framework, FW; framework region, FR) provide structure support function. By grafting the CDRs to an appropriately selected FW, some or all of the antibody-binding activity can be transferred to the resulting recombinant antibody.

CDR grafting is the selection of a most appropriate human antibody acceptor for the graft. Various strategies have been developed to select human antibody acceptors with the highest similarities to the amino acid sequences of donor CDRs or donor FW, or to the donor structures. All these “best fit” strategies, while appearing very rational, are in fact based on one assumption, i.e., a resulting recombinant antibody that is most similar (in amino acid sequence or in structure) to the original antibody will best preserve the original antigen binding activity.

Not all amino acids in the CDRs are involved in antigen binding. Thus, it has been proposed that the grafting of only those residues that are critical in antigen-antibody interaction—the so-called specificity determining residues grafting (SDR-grafting)—will further increase the content of human antibody sequences in the resulting recombinant antibody. The application of this strategy requires information on the antibody structure as well as antibody-antigen contact residues, which are quite often unavailable. Even when such information is available, there is no systematic method to reliably identify the SDRs, and SDR-grafting remains so far mostly at the basic research level.

Recently, a strategy called “human framework shuffling” has been developed. This technique works by ligating DNA fragments encoding CDRs to DNA fragments encoding human FR1, FR2, FR3, and FR4, thus generating a library of all combinations between donor CDRs and human FRs. Methods for making human-adapted antibodies based on molecular structures, modeling and sequences for human engineering of antibody molecules are disclosed in U.S. Pat. No. 8,748,356, which is incorporated by reference for these methods.

Also disclosed is a pharmaceutical composition comprising a molecule disclosed herein in a pharmaceutically acceptable carrier. Also disclosed is a method for targeted ubiquitination of target receptors in a subject that involves administering to the subject a therapeutically effective amount of a disclosed pharmaceutical composition. Also disclosed is a kit comprising a bispecific antibody disclosed herein.

Also disclosed is an expression vector comprising an isolated nucleic acid encoding a bispecific antibody disclosed herein operably linked to an expression control sequence. Also disclosed is a cell comprising the disclosed expression vector. The cell can be a primary cell, transformed cell, cell line, or the like. In some cases, the cell is a mammalian cell line. In some cases, the cell is a non-mammalian cell line. For example, the cell can be a bacteria or insect cell line.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates an embodiment of a bi-specific antibody for outside-in ubiquitination and membrane clearance of target receptors.

FIG. 2A is a schematic depicting how DVL crosslinks ZNRF3 & Frizzled ICDs to facilitate ubiquitination of Frizzled. FIG. 2B illustrates R-spondin mediated crosslinking of the ZNRF3 and LGR5 ECDs drives membrane clearance of LGR5 and restores Frizzled levels. FIG. 2C is a bar graph showing results of a luciferase assay performed in SuperTopFlash 293T cells to measure activity of Wnt3a alone, or Wnt3a+R-spondin 2 (Rspo2).

FIG. 3A is a schematic of ligand-inducible system for ZNRF3-mediated ubiquitination of Frizzled. FIG. 3B shows binding affinity and yeast display of ZNRF3-specific scFv. Surface plasmon resonance was used to determine the binding affinity of a ZNRF3-specific scFv. Yeast display & ZNRF3 binding of the scFv was detected by flow cytometry.

FIG. 3C shows possible models describing the relationship between ZNRF3-Frizzled distance and ubiquitination efficiency. FIG. 3D shows possible models depicting the relationship between binding affinity and ubiquitination efficiency.

FIG. 4A shows eight different human TMULs that are screened for their ability to ubiquitinate twelve different therapeutically important human receptors. FIG. 4B illustrates that to recruit each TMUL with each receptor, chimeric proteins are created in which the extracellular TMUL PA domain is replaced with a BC2 nanobody, and co-transfected receptors are tagged with the BC2 peptide epitope.

FIG. 5 shows Nanobody B8 targeting the ECD of the transmembrane E3 ligase GRAIL (aka RNF128).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the lgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′) 2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “bispecific antibody” refers to an antibody having two different antigen-binding regions defined by different antibody sequences. This can be understood as different target binding but includes as well binding to different epitopes in one target.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, lgG, IgM and lgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′) 2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

The term “isolated nucleic acid” refers to a polynucleotide of genomic, cDNA, synthetic, or natural origin or some combination thereof, which (1) is not associated with the cell in which the “isolated nucleic acid” is found in nature, or (2) is operably linked to a polynucleotide to which it is not linked in nature.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-a-amino butyric acid, L-y-amino butyric acid, L-a-amino isobutyric acid, L-E-amino caproic acid, 7-amino heptanoic acid, L-aspartic acid, L-glutamic acid, N-E-Boc-N-a-CBZ-L-lysine, N-E-Boc-N-a-Fmoc-L-lysine, L-methionine sulfone, L-norleucine, L-norvaline, N-a-Boc-N-ÖCBZ-L-ornithine, N-o-Boc-N-a-CBZ-L-ornithine, Boc-p-nitro-L-phenylalanine, Boc-hydroxyproline, and Boc-L-thioproline.

The term “protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” is meant a molecule that is produced as a result of transcription of a gene. Gene products include RNA molecules transcribed from a gene, as well as proteins translated from such transcripts.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Disclosed are compositions and methods for targeted ubiquitination of target receptors. In particular, bi-specific antibodies are disclosed that are able to simultaneously bind a target receptor rand a TMUL. Provided are fusion polypeptides capable of forming a bispecific engineered antibody that is able to engage target receptors and catalyze their ubiquitination by co-binding a TMUL. The engineered antibody may comprise for example, at least one scFv, at least one Fab fragment, at least one Fv fragment, etc. It may be bivalent, trivalent, tetravalent, etc. The multivalent antibodies is multispecific, e.g., bispecific, trispecific, tetraspecific, etc. The multivalent antibodies may be in any form, such as a diabody, triabody, tetrabody, etc.

### Bispecific Antibodies

Bispecific antibodies may contain a heavy chain comprising one or more variable regions and/or a light chain comprising one or more variable regions. Bispecific antibodies can be constructed using only antibody variable domains. A fairly efficient and relatively simple method is to make the linker sequence between the V_{H} and V_{L} domains so short that they cannot fold over and bind one another. Reduction of the linker length to 3-12 residues prevents the monomeric configuration of the scFv molecule and favors intermolecular VH-VL pairings with formation of a 60 kDa non-covalent scFv dimer “diabody”. The diabody format can also be used for generation of recombinant bis-pecific antibodies, which are obtained by the noncovalent association of two single-chain fusion products, consisting of the VH domain from one antibody connected by a short linker to the VL domain of another antibody. Reducing the linker length still further below three residues can result in the formation of trimers (“triabody”, about 90 kDa) or tetramers (“tetrabody”, about 120 kDa). For a review of engineered antibodies, particularly single domain fragments, see Holliger and Hudson, 2005, Nature Biotechnology, 23:1126-1136. All of such engineered antibodies may be used in the fusion polypeptides provided herein.

Peptide linkers (−) suitable for production of scFv antibodies are described in Kumada Y, et al. Biochemical Engineering Journal. 2007 35 (2): 158-165; Albrecht H, et al. J Immunol Methods. 2006 310 (1-2): 100-16; Feng J, et al. J Immunol Methods. 2003 282 (1-2): 33-43; Griffiths A D, et al. Curr Opin Biotechnol. 1998 9 (1): 102-8; Huston J S, et al. Methods Enzymol. 1991 203:46-88; Bird R E, et al. Science. 1988 242 (4877): 423-6; Takkinen K, et al. Protein Eng. 1991 4 (7): 837-41; Smallshaw J E, et al. Protein Eng. 1999 12 (7): 623-30; Argos P. J Mol Biol. 1990 211 (4): 943-58; and Whitlow M, et al. Protein Eng. 1993 6 (8): 989-95, which are hereby incorporated by reference for the teachings of these linkers and methods of producing scFv antibodies against different targets using various linkers.

Tetravalent Tandab® may be prepared substantially as described in WO 1999/057150 A3 or US2006/0233787, which are incorporated by reference for the teaching of methods of making Tandab® molecules.

The antigen recognition sites or entire variable regions of the engineered antibodies may be derived from one or more parental antibodies directed against any antigen of interest (e.g., target receptor ECD or TMUL ECD). The parental antibodies can include naturally occurring antibodies or antibody fragments, antibodies or antibody fragments adapted from naturally occurring antibodies, antibodies constructed de novo using sequences of antibodies or antibody fragments known to be specific for an antigen of interest. Sequences that may be derived from parental antibodies include heavy and/or light chain variable regions and/or CDRs, framework regions or other portions thereof.

In some cases, the TMUL antigen-binding fragment of the disclosed bi-specific antibody is a ZNRF3-specific scFv “Z6” having the amino acid sequence

(SEQ ID NO: 1)
ASQVQLVQSGAEVKNPGASVKVSCKASGYAFTSYGISWVRQAPGQGLEW
MGWISAYTRNTNYAQKFQGRVTLTTDTSTSTAYMELRSLRSDDTAIYYC
ARDARYSLGVGAFDVWGQGTMVTVSSGILGSGGGGSGGGGSGGGGSETT
LTQSPAFMSATPGDKVSISCKASRDIDDDLNWYQQKPGEAAISIIQEAT
TLVPGIPPRFSGSGYGTDFTLTINNIESEDAAYYFCLQHDDVPYTFGQG
TKLEIKSGIL.

In some cases, the TMUL antigen-binding fragment of the disclosed bi-specific antibody is an RNF43-Specific scFv having the amino acid sequence

(SEQ ID NO: 2)
ASQITLKESGPTLVKPTQTLTLTCSFSGFSLSFSGVGVAWIRQPPGKAL
EWLALIYWDDDKRYSPSLKSRLTITKDTSKNQVVLTMTNMDPLDTATYY
CAHREWKAFGAFDIWGQGTMVTVSSGILGSGGGGSGGGGSGGGGSQPVL
TQSPSASGTPGQRVTISCSGSSSNIGSNTVNWYQQLPGTAPKLLIYSNN
QRPSGVPDRFSGSKSVTSASLAISGLQSEDEAEYYCATWDDSLNGAVFG
GGTQLTVLSGIL.

In some cases, the VAT comprises the amino acid sequence ASQVQLVQSGAEVKNPGASVKVSCKASGYAFTSYGISWVRQAPGQGLEWMGWISAYTRN TNYAQKFQGRVTLTTDTSTSTAYMELRSLRSDDTAIYYCARDARYSLGVGAFDVWGQG™ VTVSSGIL SEQ ID NO:3, or a fragment or variant thereof able to bind ZNRF3. In some cases, the V_{L}T comprises the amino acid sequence ETTLTQSPAFMSATPGDKVSISCKASRDIDDDLNWYQQKPGEAAISIIQEATTLVPGIPPRFS GSGYGTDFTLTINNIESEDAAYYFCLQHDDVPYTFGQGTKLEIKSGILSEQ ID NO: 4, or a fragment or variant thereof able to bind ZNRF3. In some cases, the V_{H}T comprises the amino acid sequence ASQITLKESGPTLVKPTQTLTLTCSFSGFSLSFSGVGVAWIRQPPGKALEWLALIYWDDDKR YSPSLKSRLTITKDTSKNQVVLTMTNMDPLDTATYYCAHREWKAFGAFDIWGQGTMVTVSS GILSEQ ID NO: 5, or a fragment or variant thereof able to bind RNF43. In some cases, the V_{L}T comprises the amino acid sequence QPVLTQSPSASGTPGQRVTISCSGSSSNIGSNTVNWYQQLPGTAPKLLIYSNNQRPSGVPD RFSGSKSVTSASLAISGLQSEDEAEYYCATWDDSLNGAVFGGGTQLTVLSGIL SEQ ID NO: 6, or a fragment or variant thereof able to bind RNF43.

In some cases, the TMUL antigen-binding fragment of the disclosed bi-specific antibody is an RNF128-Specific scFv or nanobody. In some cases, the RNF128-Specific nanobody has the amino acid sequence QVQLQESGGGLVQAGGSLRLSCAASGNISYFLIMGWYRQAPGKEREFVAAITRGSNTYYA DSVKGRFTISRDNAKNTVYLQMNSLKPEDTAVYYCAVFSTLQYHYDTGYTAYLTYWGQGT QVTVSS (SEQ ID NO:7), or a fragment or variant thereof able to bind RNF128.

In some embodiments, the RNF128-Specific nanobody can comprise a variable domain having CDR1, CDR2 and CDR3 sequences. For example, in some embodiments, the CDR1 sequence comprises the amino acid sequence NISYFLI (SEQ ID NO: 8); CDR2 sequence of the variable domain comprises the amino acid sequence EFVAAITRGSNTYY (SEQ ID NO:9); and the CDR3 sequence of the variable domain comprises the amino acid sequence AVFSTLQYHYDTGYTAYLTY (SEQ ID NO:10).

The particular length of the peptide linker (--) used to join the scFv molecules together is important in determining half-life, immunogenicity, and activity of the overall construct. In some embodiments, the linker sequence (--) is 4, 5, 6, 7, 8, 9, 10, 11, 12, 13, 14, 15, 16, 17, 18, 19, 20, 21, 22, 23, 24, 25 or more amino acids in length. In some embodiments, the linker sequence (--) comprises GGGGS (SEQ ID NO:11). In some cases, the linker comprises 2, 3, 4, 5, or more GGGGS sequences. The linker is preferably long enough to not interfere with proper folding and association of the V_{H}-V_{L} chains but not so long as to cause added immunogenicity.

Candidate engineered antibodies for inclusion in the fusion polypeptides, or the fusion polypeptides themselves, may be screened for activity using a variety of known assays. For example, screening assays to determine binding specificity are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6.

In some embodiments, the bispecific antibody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Bispecific antibodies which have been altered will therefore remain administrable for a longer period of time with reduced or no immune response-related side effects than corresponding bispecific antibodies which have not undergone any such alteration(s). One of ordinary skill in the art will understand how to determine whether, and to what degree an antibody must be altered in order to prevent it from eliciting an unwanted host immune response.

### Pharmaceutical Composition

Also disclosed is a pharmaceutical composition comprising a disclosed molecule in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutically acceptable carriers include any and all suitable solvents, dispersion media, coatings, antibacterial and antifungal agents, isotonicity agents, antioxidants and absorption delaying agents, and the like that are physiologically compatible with a bispecific antibody of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

Pharmaceutical bispecific antibodies may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole, butylated hydroxytoluene, lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical bispecific antibodies may also comprise isotonicity agents, such as sugars, polyalcohols, such as mannitol, sorbitol, glycerol or sodium chloride in the compositions.

The pharmaceutical bispecific antibodies may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The bispecific antibodies may be prepared with carriers that will protect the bispecific antibody against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art.

Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Also disclosed is the use of a disclosed bispecific antibody for use as a medicament for the treatment of various forms of cancer, including metastatic cancer and refractory cancer.

### Methods of Treatment

Also disclosed is a method for treating a diseases associated with cell surface receptors in a subject by administering to the subject a therapeutically effective amount of the disclosed pharmaceutical composition. Examples of diseases that can be treated include cancer, autoimmune disease, diabetes, neurological disorders, chronic viral infections, bacterial infections, parasitic infections, Alzheimer's disease, heart disease.

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for the disease. Thus, the method can further comprise identifying a subject at risk for the disease prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule containing lenalidomide administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

The disclosed bispecific antibodies may also be administered in combination therapy, i.e., combined with other therapeutic agents relevant for the disease or condition to be treated. Accordingly, in one embodiment, the antibody-containing medicament is for combination with one or more further therapeutic agent.

A number of embodiments of the invention have been described. Nevertheless, it will be understood that various modifications may be made without departing from the spirit and scope of the invention. Accordingly, other embodiments are within the scope of the following claims.

### EXAMPLES

The ubiquitination proteasome pathway is an evolutionarily conserved cellular waste disposal system that mediates protein degradation through the coordinated actions of E1 enzymes, E2 enzymes, and E3 ligases. This process begins when an E1 enzyme activates ubiquitin and attaches it to an E2 enzyme. An E3 ligase then binds to both the E2-ubiquitin conjugate and to a protein substrate, and this interaction facilitates ubiquitin transfer from E2 to substrate. E3 ligases generally control target specificity because many E2 enzymes are promiscuous and only require a substrate to be brought within close proximity for ubiquitin transfer to occur.

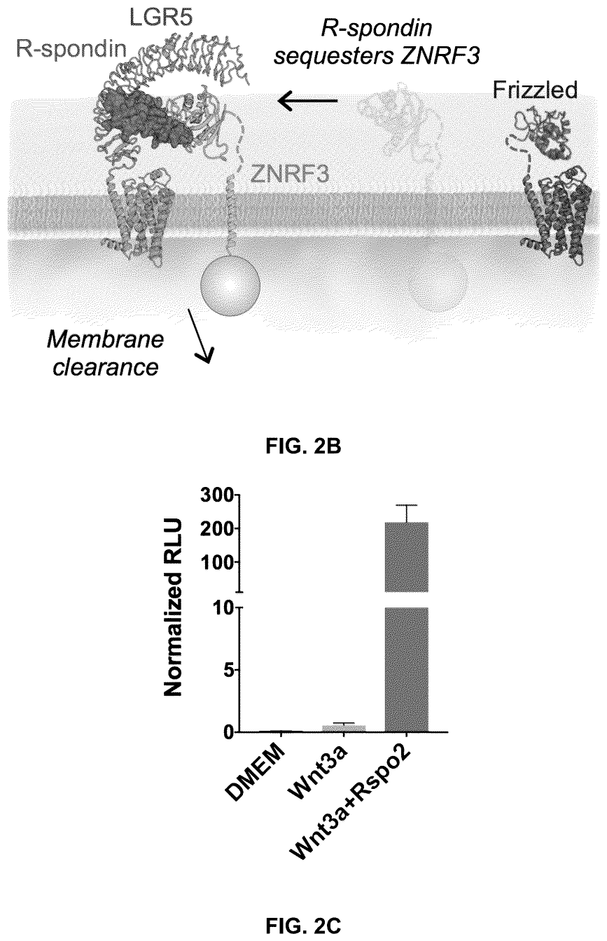

As a proof-of-principle, ZNRF3, a TMUL that guides cell fate decisions by facilitating membrane clearance of the Wnt signaling receptor Frizzled, was examined. ZNRF3 possesses an extracellular PA domain, a transmembrane domain, and an intracellular RING E3 domain. Frizzled and ZNRF3 are brought together through mutual interactions between each of their ICDs and the cytosolic protein Dishevelled (DVL), and this co-localization enables ZNRF3 to catalyze ubiquitination of the Frizzled ICD (FIG. 2A) (Hao H-X, et al. Nature. 2012 485 (7397): 195-200). ZNRF3-mediated downregulation of Frizzled may be overcome by the secreted ligand R spondin, which sequesters ZNRF3 by cross-linking its PA domain to the extracellular domain (ECD) of the co-receptor LGR5 (FIG. 2B) (Hao H-X, et al. Nature. 2012 485 (7397): 195-200; Wang D, et al. Genes Dev. 2013 27 (12): 1339-1344). Therefore, R spondin functions as a molecular “toggle switch” that redirects ZNRF3 to drive E3-dependent membrane clearance of LGR5 instead of Frizzled (Hao H-X, et al. Nature. 2012 485 (7397): 195-200). To demonstrate the remarkable potency with which ZNRF3 inhibits Frizzled, a signaling assay (SuperTopFlash™) was conducted using a 293T Wnt reporter cell line known to express ZNRF3, which revealed that Wnt has a nearly undetectable effect over background in the absence of R-spondin (FIG. 2C).

In addition to ZNRF3, there are many other important PA domain-containing TMULs encoded by the human genome. For example, the TMUL GRAIL promotes T cell tolerance by downregulating the receptors CD83, CD40L and CD151 (Anandasabapathy N, et al. Immunity. 2003 18 (4): 535-547), the TMUL RNF149 attenuates cell growth by downregulating cytosolic BRAF proteins (Hong S-W, et al. J Biol Chem. 2012 287 (28): 24017-24025), and the TMUL RNF167 influences synaptic transmission by downregulating AMPA receptors (Lussier M P, et al. Proc Natl Acad Sci. 2012 109 (47): 19426-19431). Collectively, studies of the human TMUL repertoire highlight the diversity in function, tissue distribution, and cellular substrate recognition exhibited by these unusual proteins.

### Example 1: How do TMULs Target Cellular Substrates?

Example 1 was designed to deeply interrogate fundamental TMUL biology and answer the question: how do TMULs bind and subsequently modify their substrates? Despite the emerging importance of TMULs in processes ranging from stem cell renewal (Hao H-X, et al. Nature. 2012 485 (7397): 195-200) to immune tolerance (Anandasabapathy N, et al. Immunity. 2003 18 (4): 535-547), there still is not a clear picture of the structural and biochemical parameters that control TMUL function. This lack of mechanistic information limits the ability to interpret numerous TMUL-regulated biological processes and obscures efforts to engineer biologics that hijack TMUL-mediated ubiquitination to destroy therapeutic receptor targets.

Conceptually, the ability of TMULs to convert extracellular cues into changes in intracellular effector function mirrors the behavior of classical receptor systems such as the receptor tyrosine kinases (RTKs) and cytokine receptors. For both RTKs and cytokine receptors, it has been established that extracellular docking geometry and binding kinetics directly influence downstream signaling outcomes, and detailed structure-function studies of their activation mechanisms have guided the design of antibodies6 and cytokines (Levin A M, et al. Nature. 2012 484 (7395): 529-533) with unique therapeutic properties. Consequently, the molecular determinants of TMUL-substrate recognition are elucidated. A multi-pronged approach is used: (i) visualize ZNRF3-DVL-Frizzled interactions, (ii) determine how geometry and affinity influence substrate modification, and (iii) identify which E2 enzymes couple with the ZNRF3 E3 ligase to ubiquitinate Frizzled.

### TMUL-Substrate Structure

Structural characterization of TMUL-substrate interactions are key to dissecting the molecular mechanisms by which they function. Thus far, it has not been possible to “see” how a given TMUL binds its natural substrate, and therefore how parameters such as docking geometry and interface chemistry influence ubiquitin transfer are not known. Here, x-ray crystallography is used to determine the structure of a ZNRF3-DVL-Frizzled complex. The transmembrane proteins ZNRF3 and Frizzled are expressed in insect cells, solubilized from membranes using gentle detergents, and purified by affinity and size-exclusion chromatography. In parallel, soluble DVL are purified from a bacterial expression system. Purified proteins are then used to reconstitute the ternary complexes and screened for co-crystallization using either standard protocols or the powerful lipidic cubic phase method known to facilitate membrane protein crystallization. Structures of ZNRF3-DVL or Frizzled-DVL binary complexes, or structures of smaller complexes that contain only the minimal interacting regions of ZNRF3 (residues 346-528) and DVL (DEP domain) (Jiang X, et al. Mol Cell. 2015 58 (3): 522-533) are determined.

### TMUL-Substrate Geometry

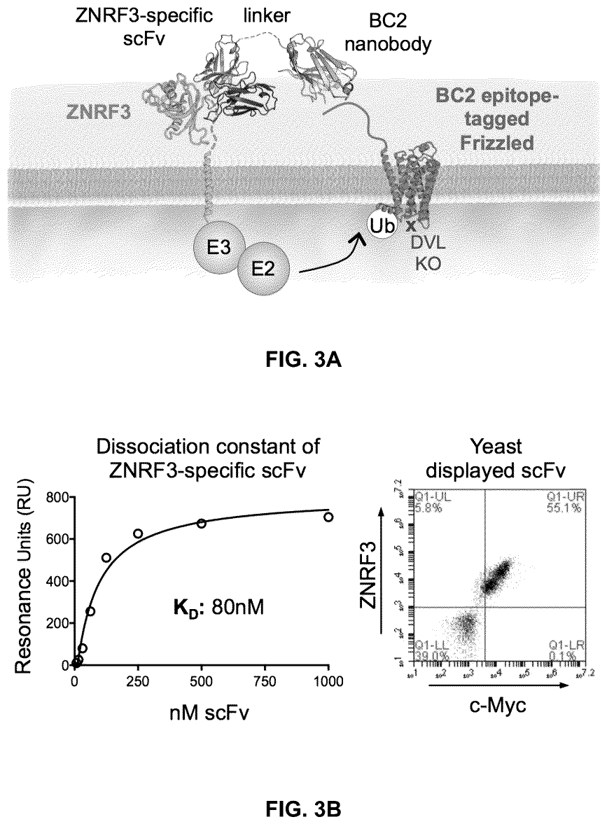

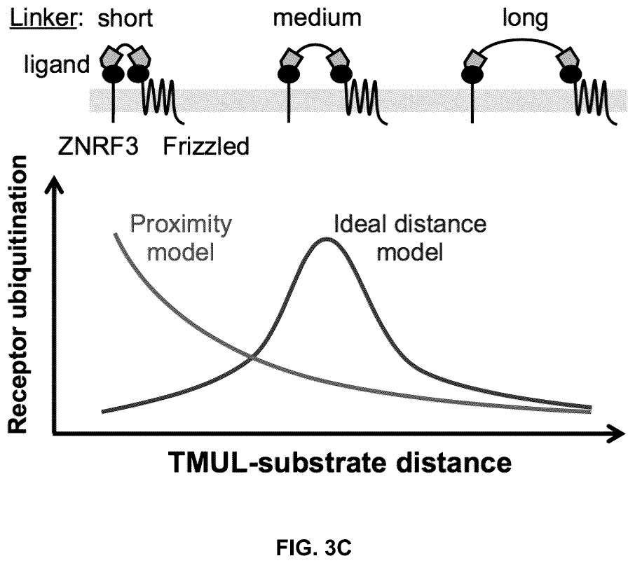

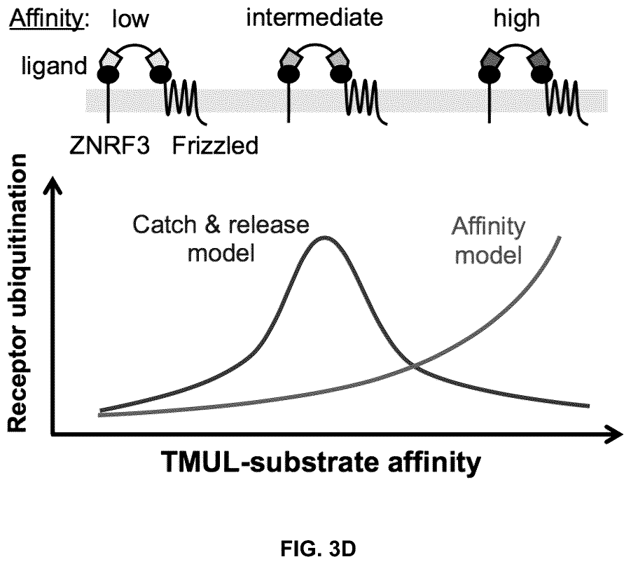

It is not presently understood how spatial and geometrical restraints control TMUL-mediated ubiquitination of cellular targets. a ligand-inducible assay is therefore developed to monitor how the intermolecular distance between the ZNRF3 and Frizzled ECDs affects ubiquitination efficiency (FIG. 3A). This assay involves the transfection of a ZNRF3-expressing cell line (293T) (Hao H-X, et al. Nature. 2012 485 (7397): 195-200) with Frizzled receptors that include (a) mutations known to ablate DVL binding (K446M, D4571, D4601) (Yu A, et al. Struct Lond Engl 1993. 2010 18 (10): 1311-1320) and (b) an extracellular BC2 peptide epitope tag (Braun M B, et al. Sci Rep. 2016 6:19211). This DVL-knockout mutation will prevent ZNRF3 from modifying the Frizzled receptors in the absence of a cross-linking ligand. The cells are then treated with a chimeric protein consisting of a ZNRF3-specific single-chain antibody variable fragment (scFv) (characterized in FIG. 3B) fused to the BC2 nanobody (Braun M B, et al. Sci Rep. 2016 6:19211) to restore ZNRF3-Frizzled interactions (FIG. 3A). To discretely vary the maximum allowable separation between ZNRF3 and Frizzled, a series of rigid helical (EAAAK) n (SEQ ID NO:12) spacers (Arai R, et al. Protein Eng. 2001 14 (8): 529-532) of known lengths are introduce between the scFv and nanobody. After transfected cells are incubated with the various scFv-nanobody fusion proteins, changes in Frizzled surface levels are assessed by immunofluorescence, and ubiquitination is monitored by western blotting to detect an increase in molecular weight.

The above experiments provide answers to important mechanistic questions about TMUL function. For example, does ZNRF3 adhere to an “ideal distance model” in which ubiquitin transfer occurs optimally at a specific separation length (FIG. 3C)? Alternatively, does ZNRF3-mediated ubiquitination follow a “proximity model” and occur most efficiently at the shortest distances (FIG. 3C)? Furthermore, identifying the optimal separation length between TMUL and substrate informs efforts to design biologics intended to redirect TMULs to ubiquitinate non-natural targets.

### Tmul-Substrate Affinity

In addition to geometrical restraints, TMULs may also have specific kinetic or affinity requirements for ubiquitin transfer. On one hand, it is possible that TMULs are purely affinity-driven such that tighter binding leads to increased ubiquitination (FIG. 3D). On the other hand, TMUL activity may follow a “catch-and-release model” in which an intermediate affinity maximizes ubiquitination rates by enabling a TMUL to let go of one target before rapidly moving on to another (FIG. 3D).

A modified version of the ligand inducible ubiquitination assay described above (FIG. 3A) is used to probe how binding affinity influences TMUL function. In this assay, the kinetics and affinity of Frizzled recruitment to ZNRF3 is precisely controlled by varying the affinity of the scFv component of the bispecific scFv-nanobody ligand (FIG. 3A). It has been determined that the ZNRF3-specific scFv binds to the ZNRF3 ECD with a Kd of 80 nM, which is in the “moderate” affinity range for an antibody-based binder (FIG. 3B). scFvs with a broad spectrum of different binding affinities are next engineered using in vitro evolution by yeast surface display. Variants with increased affinities for ZNRF3 are isolated by generating a mutant library of the scFv and performing positive selections against the ZNRF3 ECD, and variants with decreased affinity are isolated by performing negative selections against the ZNRF3 ECD. To demonstrate the feasibility of the in vitro evolution experiment, the ZNRF3-specific scFv are expressed on yeast cells and whether it binds to fluorescently labeled ZNRF3 ECDs verified using flow cytometry (FIG. 3B).

### Tmul-Associated E2 Enzymes

Many of the ˜40 human E2 enzymes have well-defined sets of cellular substrates or known biochemical requirements for target modification. However, the E2 enzymes that support substrate ubiquitination by ZNRF3 and other TMULs are presently unknown. Identification of ZNRF3-associated E2 proteins not only illuminate an essential step in the pathway, but also provide us with clues as to which additional substrates are amenable to ubiquitination by ZNRF3. We will use an in vitro ubiquitination assay to identify E2 enzymes that pair with the ZNRF3 E3 ligase to ubiquitinate Frizzled. To conduct this assay, a commercially available screen (Ubiquigent™) is adapted by combining individual E2 enzymes with purified ZNRF3 ICDs, DVL and Frizzled proteins. Western blots will then be performed to monitor Frizzled ubiquitination in each condition.

### Example 2: How to Design Extracellular Ligands to Induce Ubiquitination of Receptor ICDs?

Molecular pharmacology has traditionally focused on the discovery of agents that antagonize protein function through direct biochemical interactions. In this Example, biologics are developed that hijack the outside-in ubiquitination function of TMULs to destroy their targets outright. The strategy to knock down therapeutic receptor targets using outside-in ubiquitination is partly inspired by the natural mechanism of the R-spondin/ZNRF3 system (Hao H-X, et al. Nature. 2012 485 (7397): 195-200) (FIG. 2B), but also builds upon technological advances that have shown E3 recruitment to be a highly effective pharmacological strategy (Salami J, et al. Science 2017 355 (6330): 1163-1167). For example, drugs that induce ubiquitin-mediated proteolysis can overcome resistance that arises from protein overexpression or from mutations in active sites. These drugs would also be effective at far lower concentrations than steric inhibitors because they would not need to continuously occupy a ligand binding site (Bondeson D P, et al. Nat Chem Biol. 2015 11 (8): 611-617), and because they may be recycled after catalyzing ubiquitination. Alternatively, steric inhibitors can be linked with proteolysis targeting drugs to create a synergistic effect. Finally, E3 recruiting drugs could bind to their targets on any exposed surface, eliminating the need to identify a “perfect drug” that precisely fits into a particular active site.

To date, proteolysis targeting chimeras (PROTACs) (Sakamoto K M, et al. Proc Natl Acad Sci USA. 2001 98 (15): 8554-8559) are the most widely characterized synthetic molecules known to induce targeted E3-mediated protein knockdown. PROTACs may be either small molecules or proteins, and consist of an E3-binding moiety that is connected via a linker to a second, target-binding moiety. Thus far, a handful of small molecule PROTACs have yielded promising results in preclinical models of leukemia and prostate cancer. However, the majority of PROTACs are not effective drugs because their inherently large size is associated with poor solubility and prevents them from efficiently crossing the membrane. Additionally, PROTACs are only capable of targeting intracellular proteins that have deep druggable pockets capable of accommodating small molecule binding.

Transformative biologics are designed that reprogram TMULs to control receptor levels on the cell surface. The ability of several human TMULs to ubiquitinate a large panel of receptors associated with human diseases is evaluated. Bispecific ligands that cross-link TMULs to the ECDs of receptors identified above are engineered in order to mark the receptors for ubiquitin-mediated proteolysis. The approach enables targeting virtually any receptor, channel or transporter in its native cellular context and circumvents the need to cross the membrane, which will overcome nearly all of the obstacles that previously impeded the development of proteolysis targeting drugs.

### Determining the Breadth of TMUL Target Specificity

An important goal is the development of a technology that redirects TMULs to target unnatural substrates. A biochemical screen is conducted to identify receptors that are susceptible to TMUL-mediated ubiquitination. Ubiquitination regulates the surface levels of several receptors that contribute to human disease, including the immune checkpoint proteins PD-L1 and CD86; the innate/adaptive immune receptors IFNAR, IL-2RG, and MHCI; the HIV receptors CD4 and CXCR4; the oncogenic receptors Smo, EGFR, and HER2; and the inflammatory/autoimmune receptors TNFR1 and NDMA-R. The above 12 receptors are therefore be the first tested in the screen, both because of their translational relevance and because they have already been proven to be ubiquitinatable in natural cellular contexts.

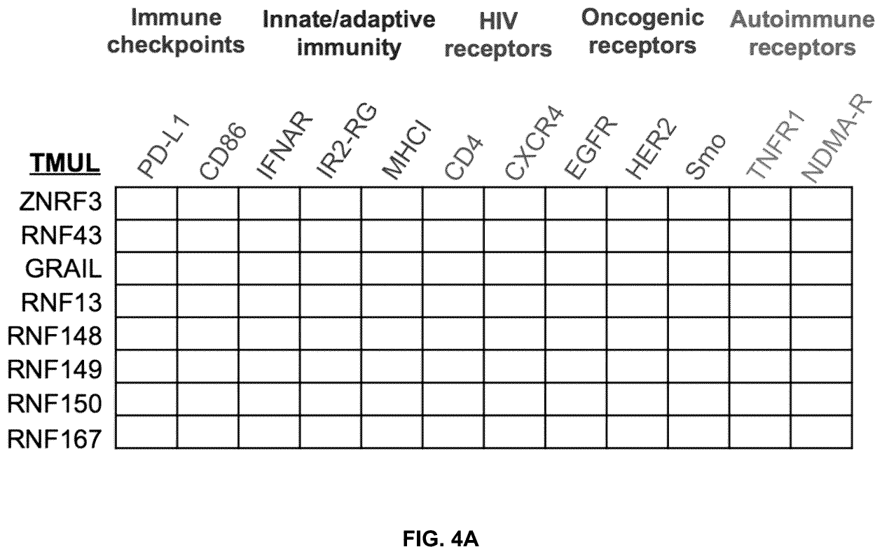

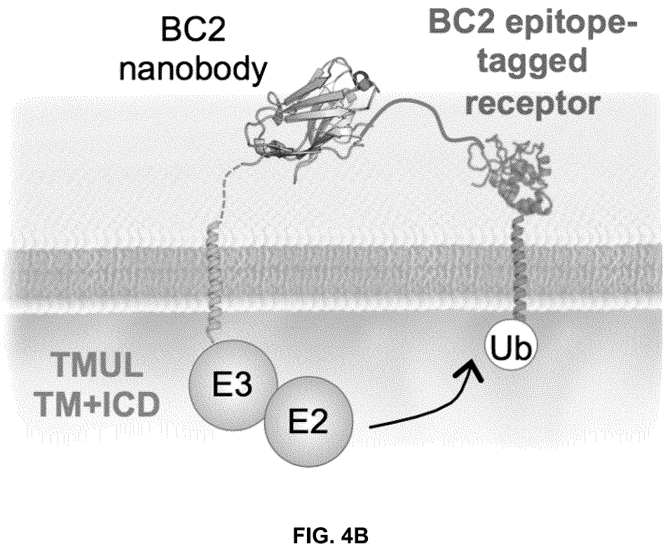

To identify receptors that are vulnerable to ubiquitination by a given TMUL, a screen is developed in which 8 different human TMULs are individually paired with the 12 receptors described above (FIG. 4A). In this assay, the extracellular PA domain of each TMUL construct is replaced with the BC2 nanobody, and the BC2-TMUL chimeras are co-transfected with receptors that have been tagged with the BC2 epitope. This arrangement brings the two proteins into close proximity on the cell surface to allow for ubiquitin transfer to occur (FIG. 4B). Expression levels are then detected by immunofluorescence, and receptor ubiquitination is tested by western blotting. The results of this screen provide important information about TMUL-substrate promiscuity, and give insight into the structure and sequences preferred by each TMUL homolog.

### Engineering Ligands to Induce TMUL-Mediated Ubiquitination of Genetically Unmodified Receptors

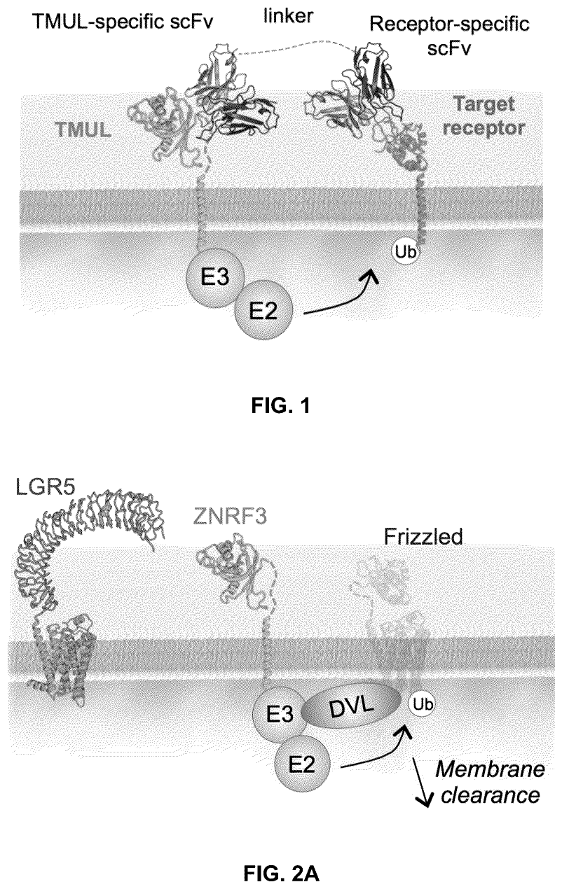

Once receptors that are susceptible to TMUL-mediated ubiquitination are identified in the synthetic system, the approach is adapted to target genetically unmodified receptors. Inducing membrane clearance of wild type receptors is a critical milestone that will demonstrate the feasibility of the method for downstream biomedical or therapeutic applications. To facilitate outside-in ubiquitination of unmodified receptors, ligands are engineered consisting of either ZNRF3-binding scFv or an scFv that recognizes one of the other 7 TMULs mentioned above fused to receptor-specific scFv via a flexible linker (FIG. 1). The bispecific ligands are then tested for their ability to induce ubiquitination and membrane clearance in 293 cells that have been transfected to express the untagged receptors, or in cell lines that endogenously express the receptor of interest. Notably, tandem scFvs in the propose format have been successfully utilized as cancer therapies (Przepiorka D, et al. Clin Cancer Res Off J Am Assoc Cancer Res. 2015 21 (18): 4035-4039), indicating that the molecules are viable for translational studies.

Tuning receptor expression levels by altering ligand affinity and geometry.

Designer ligands are created that can fine tune receptor levels on the cell surface. To achieve this goal, the mechanistic information obtained above is harnessed to adjust various aspects of the tandem scFvs so that a range of functional outcomes is achieved. For example, by varying affinity and linker length in accordance with the findings, biologics are createed that catalyze ubiquitination at different rates to stabilize “low”, “medium” and “high” receptor expression levels. Such an approach would be especially valuable in systems where complete inhibition is toxic, or where receptor signaling becomes pathogenic when elevated over a certain threshold.

### Example 3

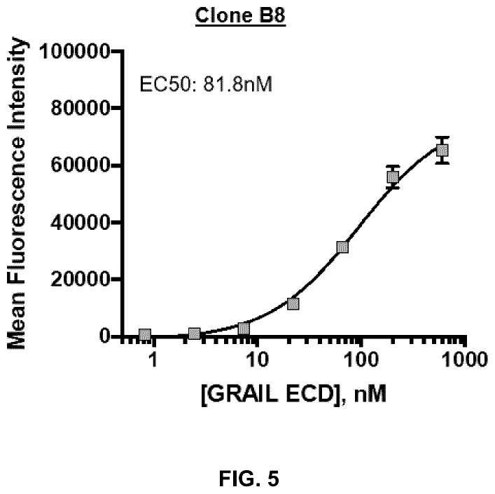

FIG. 5 shows Nanobody B8 targeting the ECD of the transmembrane E3 ligase GRAIL (aka RNF128). The amino acid sequence for Nanobody B8 is provided below:

(SEQ ID NO: 7)
QVQLQESGGGLVQAGGSLRLSCAASGNISYFLIMGWYRQAPGKEREFVA
AITRGSNTYYADSVKGRFTISRDNAKNTVYLQMNSLKPEDTAVYYCAVF
STLQYHYDTGYTAYLTYWGQGTQVTVSS.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for degrading a membrane-bound protein in a cell, comprising contacting the cell with a composition comprising a bi-specific antibody, wherein the bi-specific antibody comprises an antibody fragment specific for a transmembrane E3 ubiquitin ligase (TMUL) and an antibody fragment specific for the membrane-bound protein.

2. The method of claim 1, wherein the antibody fragment specific for TMUL is an scFv fragment or VHH fragment.

3. The method of claim 1, wherein the antibody fragment specific for the target cell surface receptor is an scFv fragment or VHH fragment.

4. The method of claim 1, comprising the following formula:
V_{L}R-V_{H}R-V_{L}T-V_{H}T,
V_{H}R-V_{L}R-V_{H}T-V_{L}T,
V_{L}R-V_{H}R-V_{H}T-V_{L}T,
V_{H}R-V_{L}R-V_{L}T-V_{H}T,
V_{H}R-V_{H}T,
V_{H}T-V_{H}R,
V_{H}R-V_{L}T-V_{L}T,
V_{H}R-V_{L}T-V_{H}T,
V_{H}R-V_{L}R-V_{H}T, or
V_{L}R-V_{H}R-V_{H}T,
wherein “V_{H}T” is a heavy chain variable domain specific for the TMUL;
wherein “V_{L}T” is a light chain variable domain specific for the TMUL;
wherein “V_{L}I” is a light chain variable domain specific for a target cell surface receptor;
wherein “V_{H}I” is a heavy chain variable domain specific for the target cell surface receptor;
wherein “-” consists of a peptide linker or a peptide bond; and
wherein the target cell surface receptor does not comprise an R-spondin protein.

5. The method of claim 4, wherein the V_{L}R and the V_{H}R have dimerized to form an antigen binding site for the target cell surface receptor, and wherein the V_{H}T and the V_{L}T have dimerized to form an antigen binding site for the TMUL.

6. The method of claim 1, wherein the TMUL is selected from the group consisting of include ZNRF3, RNF43, GRAIL, RNF13, RNF148, RNF149, RNF150, and RNF167.

7. The method of claim 1, wherein the bispecific antibody has undergone an alteration to render it less immunogenic when administered to humans.

8. The method of claim 7, wherein the alteration comprises one or more techniques selected from the group consisting of chimerization, humanization, CDR-grafting, deimmunization, and mutation of framework amino acids to correspond to the closest human germline sequence.
